(19) United States
(12) Plant Patent Application Publication
(10) Pub. No.: US 2002/0157161 P1
(43) Pub. Date: Oct. 24, 2002

(54) PETUNIA PLANT NAMED 'DANCASOFT'

(76) Inventor: Gabriel Danziger, Nir-Zvi (IL)

Correspondence Address: FOLEY & LARDNER, 3000 K Street, N.W., Suite 500, Washington, DC, 20007-5109, US

(21) Appl. No.: 09/838,231

(22) Filed: Apr. 20, 2001

(51) Int. Cl. A01H 5/00

(52) U.S. Cl. PLT/356

(57) ABSTRACT

A new and distinct cultivar of Petunia plant named ‘Dancasoft’, as described and illustrated, and particularly characterized by the combined features of pastel pink flower color, single flower type, medium flower size, early year round flowering time, spreading, well-branched vigorous growth habit, good weather tolerance, suitable for hanging baskets and bedding.

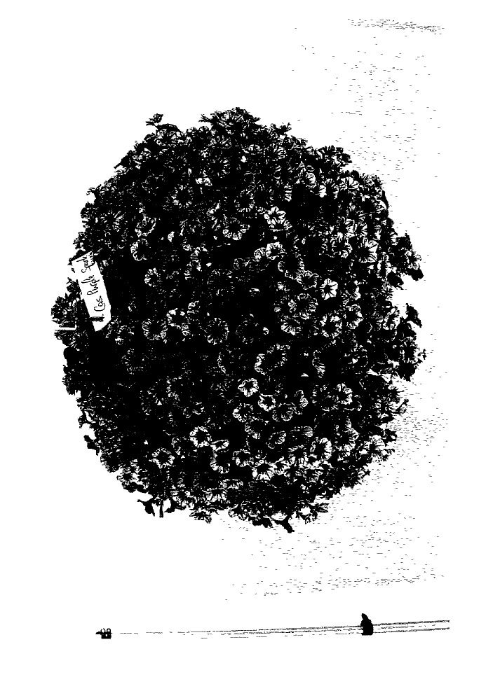

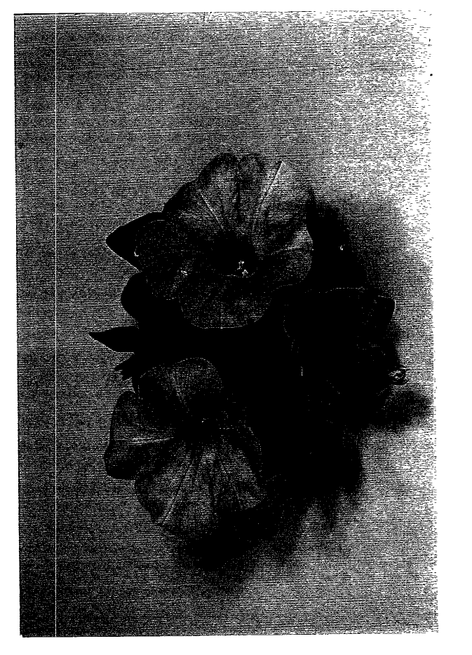

### BACKGROUND OF THE INVENTION

The present invention comprises a new and distinct cultivar of petunia, known by the cultivar name ‘Dancasoft’.

‘Dancasoft’ was originated from a hybridization made by the inventor in a controlled breeding program in Mishmar Hashiva Israel in 1998. The female was Chipper ® by Danziger. The male parent of ‘Dancasoft’ was ‘CV. 322’.

‘Dancasoft’ was discovered and selected as one flowering plant within the progeny of the stated cross by or under the supervision of the inventor Gabriel Danziger in May 1998 in a controlled environment in Mishmar Hashiva, Israel.

The first act of asexual reproduction of ‘Dancasoft’, was accomplished when vegetative cuttings were taken from the initial selection in June 1995 in a controlled environment in Mishmar Hashiva, Israel by a technician working under formulations established and supervised by the inventor. Horticultural examination of selected units initiated in September 1998 has demonstrated that the combination of characteristics of ‘Dancasoft’, as herein disclosed, are firmly fixed and are retained through successive generations of asexual reproduction.

‘Dancasoft’ has not been observed under all possible environmental conditions. The phenotype may vary somewhat with variations in environment such as temperature, light intensity and day length, however, without any variations in genotype. The following observations, measurements and comparisons describe the plants grown in Mishmar Hashiva, Israel under greenhouse conditions which approximate those generally used in commercial practice.

### BRIEF DESCRIPTION OF THE INVENTION

The following traits have been repeatedly observed and are determined to be basic characteristics of ‘Dancasoft’ which in combination distinguish this petunia as a new and distinct cultivar:

1. pastel pink flower color;

2. single flower type;

3. medium flower size;

4. early, year round flowering time;

5. spreading, well branched, vigorous growth habit;

6. good weather tolerance; and

7. suitable for hanging baskets and bedding.

Of the many commercial cultivars known to the present Inventor, the most similar in comparison to ‘Dancasoft’ is Chipper ® by Danziger “Dan” Flower Farm.

In comparison to Chipper ®, ‘DANCASOFT’ has brighter flower color, more upright growth habit, earlier flowering time, more flowers, and a longer flowering season.

### BRIEF DESCRIPTION OF THE DRAWING

The accompanying photographic drawings show typical flower and foliage characteristics of ‘Dancasoft’ with the color being as true as possible with illustrations of this type. In the following description color references are made to The Royal Horticultural Society Color Chart. The color values were determined between 10:00 a.m.-12:00 p.m. in October 2000 under full sunlight at Mishmar Hashiva, Israel.

Classification:

Botanical.—A hybrid of petunia Petunia hybrida

Commercial.—Petunia cultivar Trauma Petunia

Asexual Reproduction.—By vegetative propagation

Plant:

Growth Habit.—Trailing vigorously

Plant Height.—6-8 inch

Spreading Area.—30 inch

Blooming Period.—All year round in moderate climate

Stem:

Thickness.—2 mm

Color.—Yellow-Green 145-A

Pubescence.—Yes

Branching.—Freely

Length of interned.—20 mm

Leaf:

Arrangement on stem.—Alternate

Shape.—Elliptic

Size.—Length 50 mm

Thickness.—0.2 mm

Color.—Green 137 B

Markings.—None

Pubescence.—Yes

Flower:

Orientation at opening.—Upright

Type.—Single

Shape.—Regular, rounded, corolla tube

Diameter.—45 mm

Upper surface color.—Red-Purple 65 A

Under surface color.—Red-Purple 65 D

Peduncle Length.—15 mm

Peduncle Color.—Yellow-Green. 144 B

Reproductive Organs:

Pistil Number.—1

Pistil Color.—Yellow

Stamen Number.—5

Anther Color.—Yellow-Orange 19 B

Filament Color.—Yellow-Green 145 D

Pest/Disease Resistance: No observations made

I claim:

1. A new and distinct cultivar of Petunia plant named ‘Dancasoft’, as described and illustrated.
